(19) United States
(12) Patent Application Publication
(10) Pub. No.: US 2026/0240506 A1
(43) Pub. Date: Aug. 20, 2026

(54) PROCESSING DEVICE, NON-TRANSITORY COMPUTER-READABLE MEDIUM, AND MONITORING SYSTEM

(71) Applicant: NIHON KOHDEN CORPORATION, Saitama (JP)

(72) Inventors: Daisuke HORIGUCHI, Saitama (JP); Mitsuhiro OURA, Saitama (JP); Risa KISHI, Saitama (JP)

(73) Assignee: NIHON KOHDEN CORPORATION, Saitama (JP)

(21) Appl. No.: 19/529,927

(22) Filed: Feb. 4, 2026

(30) Foreign Application Priority Data
Feb. 14, 2025 (JP) 2025-022523

(51) Int. Cl. A61B 5/00

(52) U.S. Cl. CPC A61B 5/742; A61B 5/7475

(57) ABSTRACT

An interface receives first data corresponding to a measured value of partial pressure of arterial oxygen and a measured value of arterial oxygen saturation of a subject. The interface receives second data corresponding to a value of at least one of partial pressure of arterial carbon dioxide, pH of arterial blood, body temperature, and concentration of 2,3-DPG in blood of the subject. A processor determines a rule for mutually converting values of the partial pressure of arterial oxygen and the arterial oxygen saturation of the subject based on at least one of the first data and the second data. The processor causes a visualizing device to visualize the rule.

### CROSS-REFERENCE TO RELATED APPLICATION

The present application is based on Japanese Patent Application No. 2025-022523 filed on Feb. 14, 2025, the entire contents of which are incorporated herein by reference.

### BACKGROUND

The presently disclosed subject matter relates to a processing device adapted to monitor partial pressure of arterial oxygen, that is an index for a respiratory function of a subject. The presently disclosed subject matter also relates to a non-transitory computer readable medium having stored a computer program adapted to be executed by a processor installed in the processing device. The presently disclosed subject matter also relates to a monitoring system including the processing device and a visualizing device.

In an acute medical care, for example, management of the respiratory function of a subject is an important factor. A P/F ratio is known as a typical index indicating an oxygenation capability of a patient with respiratory failure. The P/F ratio is calculated by dividing the partial pressure of arterial oxygen (PaO2) by fraction of inspired oxygen (FiO2). Japanese Patent Publication No. 2017-538553A discloses a system in which the partial pressure of arterial oxygen is also monitored in order to optimize artificial ventilation.

### SUMMARY

It is required to enable comprehension of a relationship between oxygen saturation in blood and the partial pressure of arterial oxygen for each subject.

An illustrative aspect of the presently disclosed subject matter may provide a processing device, comprising:
an interface configured to receive:
   first data corresponding to a measured value of partial pressure of arterial oxygen and a measured value of arterial oxygen saturation of a subject; and
   second data corresponding to a value of at least one of partial pressure of arterial carbon dioxide, pH of arterial blood, body temperature, and concentration of 2,3-DPG in blood of the subject; and
a processor configured to determine a rule for mutually converting values of the partial pressure of arterial oxygen and the arterial oxygen saturation of the subject based on at least one of the first data and the second data, and to cause a visualizing device to visualize the rule.

An illustrative aspect of the presently disclosed subject matter may provide a non-transitory computer-readable medium having stored a computer program adapted to be executed by a processor installed in a processing device, the computer program being configured to cause, when executed, the processing device to:
receive first data corresponding to a measured value of partial pressure of arterial oxygen and a measured value of arterial oxygen saturation of a subject;
receive second data corresponding to a value of at least one of partial pressure of arterial carbon dioxide, pH of arterial blood, body temperature, and concentration of 2,3-DPG in blood of the subject;
determine a rule for mutually converting values of the partial pressure of arterial oxygen and the arterial oxygen saturation of the subject based on at least one of the first data and the second data; and
cause a visualizing device to visualize the rule.

An illustrative aspect of the presently disclosed subject matter may provide a monitoring system, comprising:
a processing device; and
a visualizing device,
wherein the processing device includes:
   an interface configured to receive:
      first data corresponding to a measured value of partial pressure of arterial oxygen and a measured value of arterial oxygen saturation of a subject; and
      second data corresponding to a value of at least one of partial pressure of arterial carbon dioxide, pH of arterial blood, body temperature, and concentration of 2,3-DPG in blood of the subject; and
   a processor configured to determine a rule for mutually converting values of the partial pressure of arterial oxygen and the arterial oxygen saturation of the subject based on at least one of the first data and the second data, and to cause the visualizing device to visualize the rule.

Since arterial blood sampling is required to measure PaO2, it is impossible to continuously monitor values of the PaO2 and the P/F ratio. On the other hand, not only the arterial oxygen saturation (SaO2) can be continuously measured as the transcutaneous arterial oxygen saturation (SpO2), but also the oxygen dissociation curve can be used to mutually convert the values of PaO2 and SaO2. Although an S/F ratio in which a numerator of the P/F ratio is replaced with a value of SpO2 may be used as an alternative index, the oxygenation status is normally comprehended by converting the value of SpO2 to a value of PaO2. However, the oxygen dissociation curve corresponding to the relationship is based on a non-linear function that is difficult to calculate. Accordingly, an actual situation is that medical workers memorize values of representative points in the oxygen dissociation curve to perform calculation relying on his/her experience or memory in such a manner that “When the SpO2 is 90%, the PaO2 is about 60 mmHg”. In addition, the oxygen dissociation curve is generalized by using values of standard physiological parameters, and does not reflect individual situations of each subject.

According to the configuration of each of the illustrative aspects, it is possible to determine a rule that mutually converts the values of the arterial oxygen saturation and the partial pressure of arterial oxygen of the subject is determined based on the measured value obtained from the subject, and to visualize a relationship therebetween. As a result, a user can acquire a relationship between the oxygen saturation in blood and the partial pressure of arterial oxygen for each subject without depending on his/her experience or memory.

In particular, in the case where the second data is received, since a value of at least one of the partial pressure of arterial carbon dioxide, the pH of arterial blood, the body temperature, and the concentration of 2,3-DPG in blood of the subject, that are factors causing the shift of the oxygen dissociation curve, is reflected in the conversion rule, it is possible to mitigate an adverse effect that may be caused by relying on the generalized relationship.

### DESCRIPTION OF THE DRAWINGS

FIG. 1 illustrates a functional configuration of a monitoring system according to an exemplary embodiment.

FIG. 2 illustrates an oxygen dissociation curve.

FIG. 3 illustrates factors for shifting the oxygen dissociation curve.

FIG. 4 illustrates a mapping function and a reference function that are to be visualized by a visualizing device of FIG. 1

FIG. 5 illustrates a case where a variable range of the mapping function is visualized.

FIG. 6 illustrates an appearance of a user interface of FIG. 1.

FIG. 7 illustrates a case where a calibrated or updated mapping function is visualized.

FIG. 8 illustrates a case where an estimate of PaO2 is visualized based on a measured value of SpO2.

FIG. 9 illustrates a case where an error in the measured value of SpO2 is visualized.

FIG. 10 illustrates a time series of each of measure values of SpO2, estimates PaO2, values of FiO2, and estimates of P/F ratio.

### DESCRIPTION OF EMBODIMENTS

Examples of embodiments will be described in detail below with reference to the accompanying drawings.

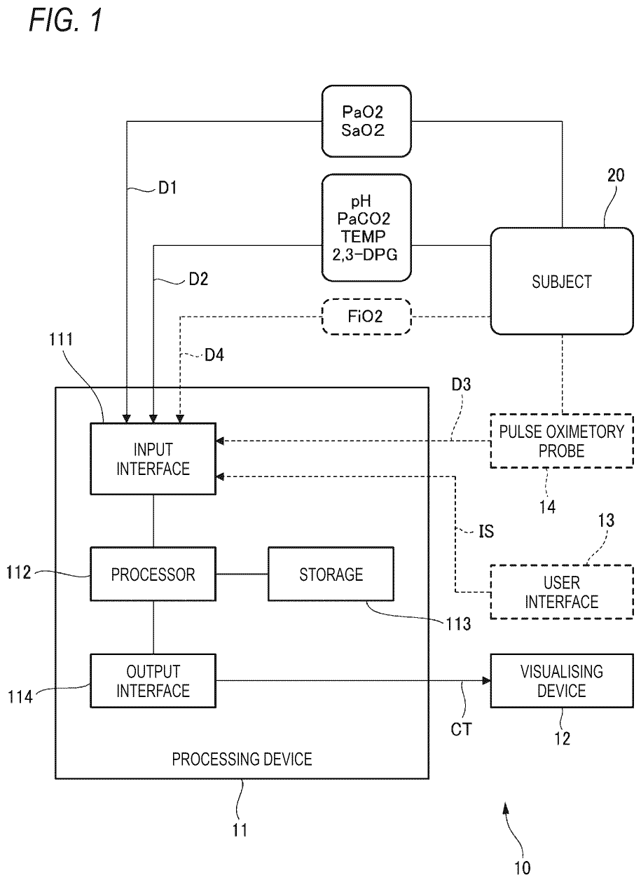

FIG. 1 illustrates a functional configuration of a monitoring system 10 according to an exemplary embodiment. The monitoring system 10 is configured to monitor partial pressure of arterial oxygen (PaO2) of a subject 20. The PaO2 is a value indicative of a partial pressure of oxygen contained in arterial blood, and is used as an index indicative of oxygenation status of blood in a lung, as the respiratory function.

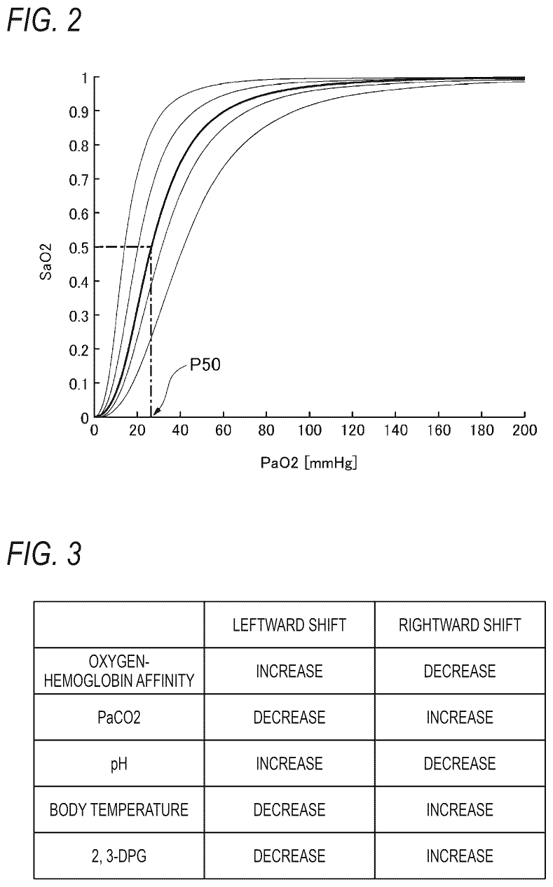

FIG. 2 illustrates the relationship between the PaO2 and an arterial oxygen saturation (SaO2). This curve is known as an oxygen dissociation curve. The SaO2 represents a proportion of hemoglobin in arterial blood that is bound to oxygen. The SaO2 may be expressed with percentages. In this example, it is illustrated as having a value from 0 corresponding to 0% to 1 corresponding to 100%. The oxygen dissociation curve is an exemplary rule that mutually converts a value of the partial pressure of arterial oxygen and a value of the arterial oxygen saturation of the subject 20.

Moreover, as illustrated by thin solid lines in FIG. 2, it is known a phenomenon in which the oxygen dissociation curve entirely shifts leftward or rightward in accordance with changes of various physiological parameters. FIG. 3 illustrates how the changes in the physiological parameters affect the shift of the oxygen dissociation curve.

Examples of physiological parameters causing the shift of the oxygen dissociation curve include affinity of oxygen and hemoglobin in arterial blood, partial pressure of arterial carbon dioxide (PaCO2), pH of arterial blood, body temperature (blood temperature), and concentration of 2,3-DPG (2,3-Diphosphoglycerate) in blood. It should be noted that the blood temperature may be estimated based on body temperature that is measured at another body site, such as body surface temperature or rectal temperature.

It is known the following equation (Hill's equation) that models the oxygen dissociation curve.

$$SaO 2 = {( PaO 2 / P 50 )}^{n} / [ 1 + {( PaO 2 / P 50 )}^{n} ] \qquad ( 1 )$$

As illustrated in FIG. 2, “P50” represents a value of PaO2 that is obtained when the SaO2 exhibits 50%. A value of 26.8 is known as a standard value of the P50. Leftward shift of the oxygen dissociation curve causes decrease of the P50, whereas rightward shift of the oxygen dissociation curve causes increase of the P50. Namely, if the P50 as a constant can be identified, a mapping function reflecting a condition of the subject 20 can be determined.

As illustrated in FIG. 1, the monitoring system 10 includes a processing device 11. An exemplary method for determining the mapping function with the processing device 11 will be described.

The processing device 11 includes an input interface 111. The input interface 111 is configured as a hardware interface that receives first data D1 corresponding to measured values of PaO2 and SaO2 of the subject 20. The measured values of PaO2 and SaO2 are obtained by subjecting arterial blood collected from the subject 20 to analysis with a blood gas analyzer (not illustrated). The first data D1 may be transmitted from the blood gas analyzer, or may be manually inputted by a user through a user interface (not illustrated).

As used herein, the expression “data corresponding to a measured value” is meant to include both a case where the data indicates the measured value itself, and a case where the data indicates a value that is obtained by applying a prescribed transformation to the measured value, an estimate of the measured value, or the like.

The first data D1 may be in the form of analog data or digital data, in accordance with the specification of the input source. In the case where the first data D1 is in the form of analog data, the input interface 111 is provided with an appropriate conversion circuit including an A/D converter. This description is similarly applied to other data that can be received by the input interface 111 described later.

The processing device 11 includes a processor 112. The processor 112 is configured to perform an operation of specifying the P50 by substituting the measured values of PaO2 and SaO2 corresponding to the first data D1 received by the input interface 111 into the Equation (1). This operation corresponds to a calibration of the oxygen dissociation curve in view of the affinity of oxygen and hemoglobin in arterial blood of the subject 20 illustrated in FIG. 3.

As illustrated in FIG. 1, the processing device 11 includes a storage 113. The processor 112 is configured to store data corresponding to the mapping function determined by the above-described operation in the storage 113. The storage 113 may be implemented by a semiconductor memory, a hard disk drive, a magnetic tape drive, or the like.

The above-described measured value of SaO2 that is inputted as the first data D1 may be replaced with a measured value of SpO2. However, it is preferable to obtain a measured value of SaO2 because it is able to be obtained together with PaO2 through a single arterial blood collection, and it represents the oxygen saturation of arterial blood more directly.

The input interface 111 of the processing device 11 is configured as a hardware interface that can also receive second data D2 corresponding to a measured value of at least one of PaCO2, pH, body temperature, and concentration of 2,3-DPG in the blood of the subject 20. The second data D2 may be received in addition to the first data D1, or may be received in place of the first data D1.

The measured values of PaCO2 and pH are obtained by subjecting arterial blood collected from the subject 20 to analysis with a blood gas analyzer (not illustrated). In this case, the second data D2 may be transmitted from the blood gas analyzer, or may be manually inputted by a user through a user interface (not illustrated).

The body temperature may be obtained by a temperature sensor (not illustrated) attached to the subject 20. The second data D2 may be transmitted from the temperature sensor, or may be manually inputted by a user through a user interface (not illustrated). It should be noted that the body temperature need not be a measured value. A value of 37° C., known as the standard temperature Ts of the blood, may be used as the value of the body temperature.

The 2,3-DPG concentration is obtained by subjecting blood collected from the subject to measurement with a measurement kit (not illustrated) using an ultraviolet spectrophotometry, for example. In this case, the second data D2 may be transmitted from the blood gas analyzer, or may be manually inputted by a user through a user interface (not illustrated). It should be noted that the 2,3-DPG concentration need not be a measured value. A value of 4.65×10⁻³ moles, known as the standard value Ds of the 2,3-DPG concentration, may be used as the value of the 2,3-DPG concentration.

The P50 in the Equation (1) may be specified on the basis of the second data D2 with the following equations.

$$P 50 = [ P 50 s ( P 50 c / P 50 s ) ( P 50 p / P 50 s ) ] ( P 50 t / P 50 s ) ( P 50 d / P 50 s ) \qquad ( 2 )$$

$$P 50 c = P 50 s + 1 \text{.273} \times {10}^{- 1} ( Pc - Pcs ) + 1.083 \times {10}^{- 4} {( Pc - Pcs )}^{2} \qquad ( 3 )$$

$$P 50 p = P 50 s - 25.535 ( ph - phs ) + 10.646 {( ph - phs )}^{2} - 1.764 {( ph - phs )}^{3} \qquad ( 4 )$$

$$P 50 t = P 50 s + 1.435 ( T - Ts ) + 4.163 \times {10}^{- 2} {( T - Ts )}^{2} + 6.86 \times {10}^{- 4} {( T - Ts )}^{3} \qquad ( 5 )$$

$$P 50 d = P 50 s + 795.63 {( D - Ds )}^{2} - 19660.89 {( D - Ds )}^{2} \qquad ( 6 )$$

where,
P50s: a standard value of P50 (26.8)
Pc: a measured value of PaCO2
Pcs: a standard value of PaCO2 (40 mmHg)
ph: a measured value of pH
phs: a standard value of pH (7.24)
T: a measured value of body temperature
Ts: a standard value of blood temperature (37° C.)
D: a measured value of concentration of 2,3-DPG in blood
Ds: a standard value of concentration of 2,3-DPG in blood (4.65×10⁻³ mol)

It should be noted that the values of the coefficients in the above Equations as well as the specific values of some physiological parameters are merely illustrative. As for the physiological parameter that is not referred to, the standard value thereof is substituted as the measured value in each Equation.

By specifying the P50 as described above, a mapping function is determined based on the various physiological parameters. The processor 112 may store data corresponding to the mapping function as determined in the storage 113.

In a case where both the first data D1 and the second data D2 are received by the input interface 111, the value of P50 that is used in determining the mapping function may be calculated by the following Equations.

$$P 50 = k 1 P 50 d 1 + k 2 P 50 d 2 \qquad ( 7 )$$

$$k 1 + k 2 = 1 \qquad ( 8 )$$

where,
P50d1: a value of P50 that is specified with the first data D1
P50d2: a value of P50 that is specified with the second data D2
   Each of k1 and k2 is a coefficient indicative of a contribution degree of each term to the P50 that is to be finally specified, and may be appropriately determined by a user. Here, k1 and k2 are positive real numbers.

The monitoring system 10 includes a visualizing device 12. The visualizing device 12 is configured to allow a user to visually recognize the mapping function determined as described above. Examples of the visualizing device 12 include a device that displays an image, a device that projects an image, and a device that prints an image. The processing device 11 may be a device independent of the visualizing device 12, or may be a part of the visualizing device 12. In the case where the processing device 11 and the visualizing device 12 are independent devices, the visualizing device 12 may be disposed or installed in a place that is remote from the processing device 11.

The processing device 11 includes an output interface 114. The processor 112 is configured to output, from the output interface 114, control data CT that causes the visualizing device 12 to visualize the mapping function as determined.

The output interface 114 is configured as a hardware interface. The control data CT may be in the form of analog data or digital data, in accordance with the specification of the visualizing device 12. In the case where the control data CT is in the form of analog data, the output interface 114 is provided with an appropriate conversion circuit including a D/A converter. This description is similarly applied to other data that can be outputted by the output interface 114 described later.

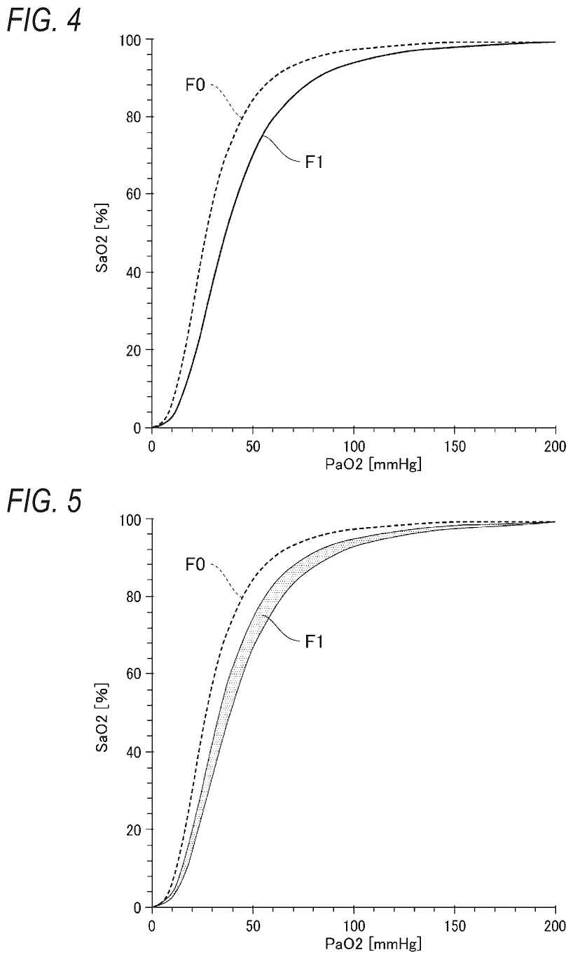

FIG. 4 illustrates a mapping function F1 that is visualized by the visualizing device 12. A dashed line represents a reference function F0. The reference function F0 corresponds to the mapping function determined in the case where the P50 is the standard value. In other words, the reference function F0 is a function that converts a value of the arterial oxygen saturation to a reference estimate of the partial pressure of arterial oxygen in a case where all of the partial pressure of arterial carbon dioxide, the pH, the body temperature, and the concentration of 2,3-DPG in blood are standard values.

Since arterial blood sampling is required to measure PaO2, it is impossible to continuously monitor values of the PaO2 and the P/F ratio. On the other hand, not only the arterial oxygen saturation (SaO2) can be continuously measured as the transcutaneous arterial oxygen saturation (SpO2), but also the oxygen dissociation curve can be used to mutually convert the values of PaO2 and SaO2. Although an S/F ratio in which a numerator of the P/F ratio is replaced with a value of SpO2 may be used as an alternative index, the oxygenation status is normally comprehended by converting the value of SpO2 to a value of PaO2. However, the oxygen dissociation curve corresponding to the relationship is based on a non-linear function that is difficult to calculate. Accordingly, an actual situation is that medical workers memorize values of representative points in the oxygen dissociation curve to perform calculation relying on his/her experience or memory in such a manner that “When the SpO2 is 90%, the PaO2 is about 60 mmHg”. In addition, the oxygen dissociation curve is a generalized curve corresponding to the above-described reference function for which the individual situation of each subject is not reflected.

According to the configuration of this exemplary embodiment, it is possible to determine a rule that mutually converts the values of the arterial oxygen saturation and the partial pressure of arterial oxygen of the subject is determined based on the measured value obtained from the subject, and to visualize a relationship therebetween. As a result, a user can acquire a relationship between the oxygen saturation in blood and the partial pressure of arterial oxygen for each subject without depending on his/her experience or memory.

In particular, in the case where the second data is received, since a value of at least one of the partial pressure of arterial carbon dioxide, the pH of arterial blood, the body temperature, and the concentration of 2,3-DPG in blood of the subject, that are factors causing the shift of the oxygen dissociation curve, is reflected into the conversion rule, it is possible to mitigate an adverse effect that may be caused by relying on the generalized relationship.

As illustrated in FIG. 4, the processor 112 of the processing device 11 may cause the visualizing device 12 to visualize the determined mapping function F1 along with the reference function F0. The reference function F0 and the mapping function F1 are visualized in a distinguishable manner. Exemplary manners include different line types, different line colors, different line widths, and presence or absence of blinking.

According to such a configuration, it is possible to cause a user to visually recognize a shift of the mapping function F1 that may occur for each subject from the reference function F0. As a result, the user can visually recognize a degree of deviation of each patient's respiratory function from the reference state.

As illustrated in FIG. 5, the processor 112 of the processing device 11 may cause the visualizing device 12 to visualize a variable range of the mapping function F1 in accordance with a variable range of the value of at least one of the PaCO2, the pH, the body temperature, and the concentration of 2,3-DPG in blood of the subject 20 associated with the second data D2.

In this example, the variable range of the mapping function F1 is visualized with a band shape, in a case where the PaCO2 is 60.6 [mmHg], the pH of arterial blood is 7.10, the body temperature is in the range of 37.5 to 39.5 [° C.], and the concentration of 2,3-DPG in blood is in the range of 3.67 to 5.01 [μmol/ml RBC].

As illustrated in FIG. 1, the monitoring system 10 may include a user interface 13. The user interface 13 may be configured to present, to a user, values of multiple parameters related to the above-described second data D2. In other words, the user interface 13 may be a part of the visualizing device 12.

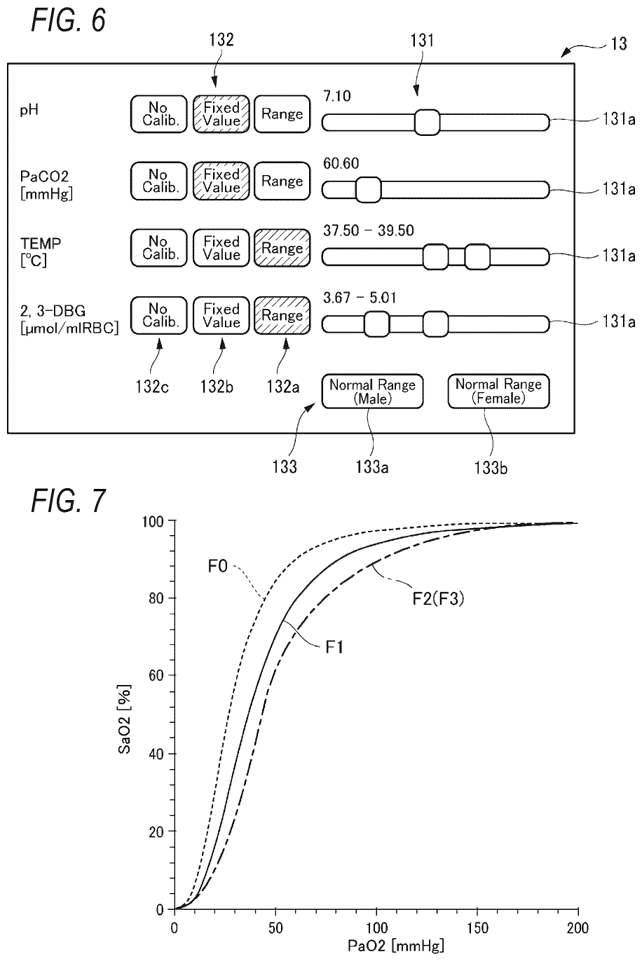

FIG. 6 illustrates an appearance of the user interface 13 having such a function. The user interface 13 includes a parameter display area 131 for presenting values of multiple parameters associated with the second data D2 to the user.

The visualization of the variable range of the mapping function F1 illustrated in FIG. 5 may be performed based on a value range of at least one of the parameters associated with the second data D2 that is automatically determined by the processor 112 of the processing device 11, or may be performed in response to a user's input of an assumed value range of at least one of the parameters to the user interface 13.

As illustrated in FIG. 6, the user interface 13 includes an input selection area 132. The input selection area 132 includes a “range” button image 132a for each of the parameters associated with the second data D2. In addition, the parameter display area 131 includes a slider image 131a adapted to specify the assumed value range of each of the parameters associated with the second data D2.

When the “range” button image 132a is brought into a selected state by a pointing device or a touch operation, two knob images appear in the slider image 131a. With an operation for sliding each of the knob images, an upper limit value and a lower limit value of the assumed value range of the corresponding parameter are specified.

The user interface 13 may include a normal range selection area 133. The normal range selection area 133 in this example includes a “male” button image 133a and a “female” button image 133b.

When the “male” button image 133a is brought into the selected state, a range of standard value that a male subject may have is automatically specified for each of the parameters associated with the second data D2. Similarly, when the “female” button image 133b is brought into the selected state, a range of standard value that a female subject may have is automatically specified for each of the parameters associated with the second data D2. In other words, a range of standard value corresponding to the gender of the subject 20 may be specified as the assumed value range. The gender is an exemplary attribute information of the subject.

Although not illustrated, an appropriate graphical user interface (GUI) may be provided to enable specifying a range of standard value in accordance with other attribute information of the subject as the assumed value range. Exemplary other attribute information includes age group, predisposition, and medical history.

As illustrated in FIG. 1, instruction data IS corresponding to the assumed value range as specified is outputted from the user interface 13. In response to reception of the instruction data IS by the input interface 111 of the processing device 11, the processor 112 identifies a variable range of the mapping function F1 based on the assumed value range, and outputs, from the output interface 114, control data CT that causes the visualizing device 12 to visualize the range as identified.

Acquisition of the measured values for all of the parameters associated with the second data D2 may be sometimes more difficult than acquisition of SaO2 and PaO2 associated with the first data D1. According to the above configuration, however, even when all of the parameters associated with the second data D2 are not obtained, it is possible to visualize a variable range of the mapping function F1 in accordance with factors for shifting the oxygen dissociation curve that is identified for each subject.

Particularly in the case where the user can input an assumed value for the variable range of at least one of the parameters associated with the second data D2, it is also visualized a condition that the mapping function F1 is varied in response to the inputted value. Accordingly, it is possible to provide a function for simulating changes that may occur in the patient's respiratory function in accordance with the factors for shifting the oxygen dissociation curve.

Particularly in the case where the normal range in accordance with the subject's attribute is specified as the variable range of at least one of the parameters associated with the second data D2, it is also visualized a condition that the mapping function F1 is varied in accordance with the attribute. Accordingly, it is possible to provide a function for simulating changes that may occur in the respiratory function differently in accordance with the subject's attribute.

As illustrated in FIG. 6, the input selection area 132 includes a “fixed value” button image 132b and a “no calibration” button image 132c for each of the parameters associated with the second data D2.

By bringing the “fixed value” button image 132b into the selected state, a single knob image is displayed on the slider image 131a. With an operation for sliding the knob image, a certain assumed value of the corresponding parameter can be specified.

By bringing the “no calibration” button image 132c into the selected state, it is cancelled a condition that the assumed value of the corresponding parameter is allowed to be specified, so that the value used in the determination of the mapping function F1 is validated.

The instruction data IS corresponding to the certain assumed value as specified is outputted from the user interface 13, so that the influence of the assumed value is reflected into the mapping function F1 that is to be visualized in the visualizing device 12. Even in this case, it is visualized a condition that the mapping function F1 is varied in accordance with the variation of at least one of the parameters associated with the second data D2. Accordingly, it is possible to provide a function for simulating changes that may occur in the patient's respiratory function in accordance with the factors for shifting the oxygen dissociation curve.

In a case where the assumed value range is canceled for all of the parameters associated with the second data D2, as illustrated in FIG. 7, a calibrated mapping function F2 without a range is visualized in the visualizing device 12. The calibrated mapping function F2 may be visualized along with at least one of the reference function F0 and the mapping function F1.

Even in this case, it is visualized a condition that the mapping function F1 is varied in accordance with the variation of at least one of the parameters associated with the second data D2. Accordingly, it is possible to provide a function for simulating changes that may occur in the patient's respiratory function in accordance with the factors for shifting the oxygen dissociation curve.

As illustrated in FIG. 1, the monitoring system 10 may include a pulse oximetry probe 14. The pulse oximetry probe 14 is attached to the body of the subject 20. The pulse oximetry probe 14 has a well-known configuration for measuring the SpO2 of the subject 20.

Specifically, the pulse oximetry probe 14 includes multiple light sources that emit light beams having wavelengths at which light absorbing properties of oxygenated hemoglobin are different. For example, a first light beam having a central wavelength in the red region, and a second light beam having a central wavelength in the infrared region are irradiated onto a living tissue including the artery of the subject 20. The pulse oximetry probe 14 includes a light detecting element. The light detecting element is configured to detect light quantity of each of the first light beam and the second light beam that have passed through the living tissue, and to output a signal corresponding to the light quantity.

The light attenuation A1 of the first light beam due to the arterial blood is specified from the difference between the light quantity of the first light beam emitted from the light source and the light quantity of the first light beam detected by the light detecting element. Similarly, the light attenuation A2 of the second light beam due to the arterial blood is specified from the difference between the light quantity of the second light beam emitted from the light source and the light quantity of the second light beam detected by the light detecting element. In a case where let Φ be a ratio of a light attenuation variation ΔA1 of the first light beam and a light attenuation variation ΔA2 of the second light beam caused by the pulsation of the arterial blood, SpO2 may be given by the following equation as a function of Φ.

$$SpO 2 = f ( Φ ) \qquad ( 9 )$$

The input interface 111 of the processing device 11 is configured as a hardware interface adapted to additionally receive third data D3 corresponding to the measured value of the SpO2 of the subject 20. The signal outputted from the pulse oximetry probe 14 is subjected to processing for calculating SpO2 with a pulse oximeter (not illustrated). The third data D3 may be transmitted from the pulse oximeter, or may be manually inputted by a user through a user interface (not illustrated).

The processor 112 reads out the data corresponding to the calibrated oxygen dissociation curve stored in the storage 113, and substitutes the measured value of SpO2 corresponding to the third data D3 in place of the value of SaO2 in Equation (1). As a result, an estimate of PaO2 based on the calibrated oxygen dissociation curve is calculated.

The value of PaO2 can be estimated with the oxygen dissociation curve as long as the value of SaO2 is obtained. However, since the SaO2 is a value obtained invasively, the value of PaO2 to be obtained must be intermittent. In addition, since the SaO2 is a value obtained invasively, multiple acquisitions of the PaO2 value would be a burden on the subject.

According to the above configuration, after the mapping function F1 is once determined with at least one of the first data D1 and the second data D2, by using the mapping function, it is possible to time-continuously calculate an estimate of PaO2 from the measured value of SpO2 that is non-invasively and time-continuously obtained. As a result, it is possible to realize time-continuous monitoring of the estimate of PaO2 of the subject 20 without imposing a burden associated with the invasion at the time of blood collection. In addition, the mapping function F1 for transforming the measured value of SpO2 to the estimate of PaO2 is determined through a minimum number of blood gas analyses (including zero rounds) based on the value of a physiological parameter of the subject 20 that may affect the shift of the oxygen dissociation curve. Accordingly, it is possible to obtain the estimate of PaO2 in which the physiological condition of the subject 20 is reflected, while further reducing the burden that would be imposed on the subject 20.

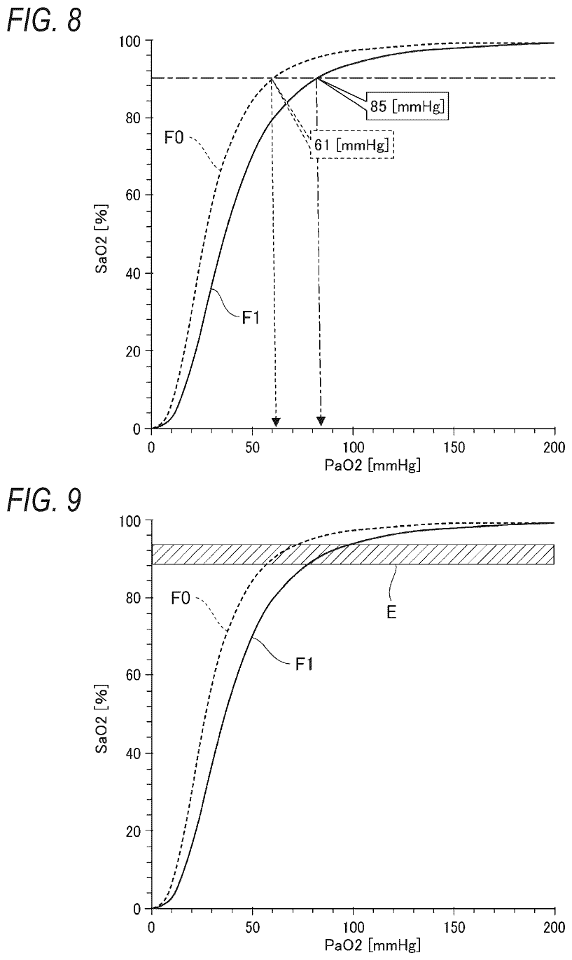

As illustrated in FIG. 8, the processor 112 of the processing device 11 may cause the visualizing device 12 to visualize the estimate of PaO2 calculated as described above. Specifically, the control data CT configured to realize visualization of the estimate in a prescribed manner is outputted from the output interface 114.

As an example, the measured value of SpO2 may be displayed along with the mapping function F1. In this example, the measured value of SpO2 is visualized as a linear line parallel to the horizontal axis. The estimate of PaO2 corresponding to the intersection point of the linear line and the curve corresponding to the mapping function F1 may be visualized in a form of a balloon so as to directly indicate a numerical value, or may be visualized in a form of a linear line parallel to the vertical axis so as to directly indicate an abscissa.

According to such a configuration, the user can visually recognize the basis of the value of PaO2 estimated from the measured value of SpO2.

In addition to or in place of the value of PaO2 that is estimated based on the mapping function F1, the processor 112 of the processing device 11 may cause the visualizing device 12 to visualize the value of PaO2 that is estimated based on the reference function F0.

According to such a configuration, the user can visually recognize a difference between the value of PaO2 estimated based on the value of the physiological parameter that is actually obtained from the subject 20, and the value of PaO2 estimated using the standard value.

As illustrated in FIG. 9, the processor 112 of the processing device 11 may cause the visualizing device 12 to visualize a marker E that indicates a measurement error of the value of SpO2 corresponding to the third data D3 along with the mapping function F1. Specifically, the control data CT configured to realize visualization of the marker in a prescribed manner is outputted from the output interface 114.

In this example, it is visualized a band-shaped marker E indicating an upper limit value and a lower limit value, that are obtained by adding or subtracting an error to or from the measured value of SpO2. The intersection point of the upper edge of the band corresponding to the upper limit value of SpO2 and the mapping function F1 corresponds to an upper limit value of the estimate of PaO2. The intersection point of the lower edge of the band corresponding to the lower limit value of SpO2 and the mapping function F1 corresponds to a lower limit value of the estimate of PaO2. These estimates may be visualized in various manners described with reference to FIG. 8.

The marker E may be visualized along with the mapping function F1 for which the variable range thereof is visualized as illustrated in FIG. 5. In addition to or in place of the mapping function F1, the marker E may be visualized along with the reference function F0.

As long as the measured value of SpO2 is inputted to the mapping function F1 in place of the measured value of SaO2, the possibility that errors may occur in the measured value of SaO2 cannot be excluded. In addition, since the SpO2 is generally presented as an integer value, the actual value of SpO2 may be distributed within a certain range. For example, in a case where a measured value of SpO2 as presented is 95%, the actual value of SpO2 is distributed in a range of 94.5% to 95.4%. Accordingly, the value of PaO2 estimated from the particular measured value of SpO2 may also be distributed within a certain range. However, according to the above configuration, it is possible to cause a user to visually recognize a variable range of the estimate of PaO2 for which such errors are considered.

It should be noted that the marker E may be updated by performing comparison with a measured value of SpO2 each time a measured value of SaO2 is discretely obtained from the subject 20.

Additionally or alternatively, the variable range of the estimate of PaO2 may be updated each time a measured value of PaO2 is discretely obtained from the subject 20.

As illustrated in FIG. 1, the input interface 111 of the processing device 11 may be configured as a hardware interface adapted to receive fourth data D4 corresponding to a value of the FiO2 of the subject 20. The value of FiO2 may be a measured value, a prescribed value, or an estimate. The fourth data D4 may be transmitted from an oxygen delivery device (not illustrated) connected to the subject 20, or may be manually inputted by a user through a user interface (not illustrated).

In this case, the processor 112 is configured to obtain an estimate of the P/F ratio (PaO2/FiO2) based on the estimate of the PaO2 obtained as described above and the value of the FiO2 corresponding to the fourth data D4. The P/F ratio is an exemplary index indicative of an oxygenation capability of the subject 20.

Since the value of PaO2 obtained through the blood gas analysis is conventionally used, the value of the P/F ratio has to be calculated intermittently. However, according to the configuration described above, since the value of P/F ratio of the subject 20 is estimated with the measured value of SpO2 that can be obtained time-continuously and non-invasively, it is possible to realize time-continuous monitoring of the oxygenation capability of the subject 20 without imposing a burden associated with the invasion at the time of blood collection.

The index indicative of the oxygenation capability of the subject 20 may not be limited to the P/F ratio as long as it is based on the PaO2. Other exemplary indices include an A-aDO2 corresponding to the difference between a PAO2 (partial pressure of alveolar oxygen calculated from the alveolar gas equation) and the PaO2, a ratio of the A-aDO2 to the PaO2 (A-aDO2/PaO2), a ratio of the PaO2 to the PAO2 (PaO2/PAO2), and an oxygenation index (OI). The OI is obtained by multiplying the value of (FiO2/PaO2) with a value of a mean airway pressure (MAP). The PAO2 may be an exemplary inspiratory oxygen information.

The processor 112 may visualize an estimate of the P/F ratio in addition to or in place of the estimate of PaO2 in each of the exemplary visualization described with reference to FIGS. 4, 5, and 7-9.

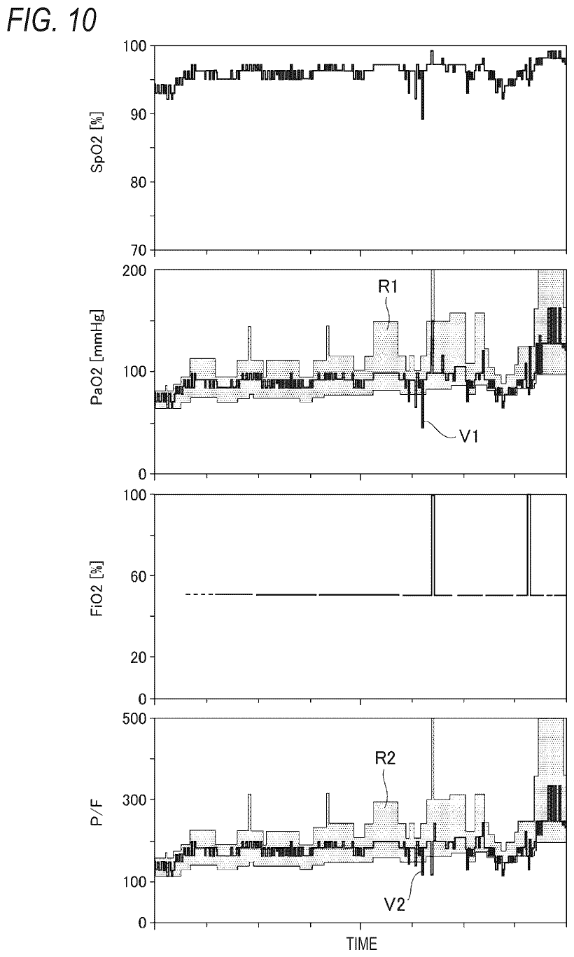

FIG. 10 illustrates changes over time (time series) of the measured values of SpO2, the estimates of PaO2, the values of FiO2, and the estimates of the P/F ratio that are described above. In addition to or in place of the visualization of the mapping function described with reference to FIGS. 4, 5, and 7-9, the processor 112 of the processing device 11 may visualize at least a time series of the estimates of PaO2 in the visualizing device 12. Specifically, the control data CT configured to realize visualization of the time series in a prescribed manner is outputted from the output interface 114. In addition to or in place of the PaO2, a time series of estimates of the P/F ratio may be visualized.

According to such a configuration, it is possible to cause a user to visually recognize time-continuous variations of the estimate of the PaO2 or the P/F ratio of the subject 20.

The processor 112 of the processing device 11 may cause the visualizing device 12 to visualize changes over time (time series) of the variable range of the estimate of PaO2 in accordance with the variable range of at least one of the parameters associated with the second data D2 described with reference to FIG. 5 (the variable range of the oxygen dissociation curve). In a case where input data involving a measurement error is used as in the case of SpO2 described with reference to FIG. 9, the “variable range of estimate” to be visualized is also affected by the measurement error. Specifically, the control data CT configured to realize visualization of the time series of the variable range in a prescribed manner is outputted from the output interface 114. In addition to or in place of the PaO2, a time series of a variable range of estimates of the P/F ratio may be visualized.

In FIG. 10, a time series V1 of the estimates of PaO2 indicated by a solid line is visualized along with a time series R1 of the variable range of the estimates such that the time series R1 surrounds the time series V1. Similarly, a time series V2 of the estimates of P/F ratio indicated by a solid line is visualized along with a time series R2 of the variable range of the estimates such that the time series R2 surrounds the time series V2.

According to such a configuration, it is possible to cause a user to visually recognize time-continuous variations of the variable range of the estimate of the PaO2 or the P/F ratio of the subject 20 due to the factors for shifting the oxygen dissociation curve.

At least one of the first data D1 and the second data D2 may be updated through periodic or non-periodic measurement. The processor 112 of the processing device 11 may be configured to update data corresponding to the mapping function F1 that is stored in the storage 113 in response to reception of at least one of the first data D1 and the second data D2 as updated by the input interface 111.

In the case where the first data D1 is updated, the processor 112 re-identifies the P50 with the method described with reference to the Equation (1), and updates the mapping function F1 with the re-identified value of the P50. The data corresponding to an updated mapping function F3 is stored in the storage 113. Thereafter, estimation of PaO2 based on the measured value of SpO2 corresponding to the third data D3 received by the input interface 111 is performed based on the updated mapping function F3.

In the case where the second data D2 is updated, the processor 112 re-identifies the P50 by the method described with reference to the Equations (2) to (6), and updates the mapping function F1 with the re-identified value of the P50. The data corresponding to an updated mapping function F3 is stored in the storage 113. Thereafter, estimation of PaO2 based on the measured value of SpO2 corresponding to the third data D3 received by the input interface 111 is performed based on the updated mapping function F3.

As illustrated in FIG. 7, the processor 112 of the processing device 11 may cause the visualizing device 12 to visualize the pre-updated mapping function F1 and the updated mapping function F3. Specifically, the control data CT that is configured to realize visualization of the mapping function F3 is outputted from the output interface 114.

According to such a configuration, the latest condition of the subject 20 can be reflected into the estimate of PaO2. In addition, it is possible to cause a user to visually recognize changes over time of the mapping function associated with the update of the physiological parameter obtained from the subject 20.

The processor 112 of the processing device 11 having various functions described above may be implemented by at least one versatile microprocessor configured to cooperate with at least one versatile memory. Examples of the versatile microprocessor include a CPU, an MPU, and a GPU. Examples of the versatile memory include a ROM, and a RAM. In this case, a computer program for executing the above-described processing may be stored in the ROM. The ROM is an exemplary non-transitory computer-readable medium having stored a computer program. The versatile microprocessor designates at least a part of the program stored in the ROM, loads the designated program in the RAM, and executes the above-described processing in cooperation with the RAM. The computer program may be pre-installed in a versatile memory, or may be downloaded from an external server device with a communication network, and then installed in the versatile memory. In this case, the external server device is an exemplary non-transitory computer-readable medium having stored therein a computer program.

The processor 112 may be implemented by at least one exclusive integrated circuitry capable of executing the above-described computer program. Examples of the exclusive integrated circuit include a microcontroller, an ASIC, and an FPGA. In this case, the above-described computer program is pre-installed in the memory element included in the exclusive integrated circuit. The memory element is an exemplary computer-readable medium having stored a computer program. The processor 112 may also be implemented by a combination of the non-exclusive microprocessor and the exclusive integrated circuitry.

Each of the configurations referenced in the foregoing description is illustrative to facilitate understanding of the presently disclosed subject matter. Each exemplary configuration may be appropriately modified or combined with another exemplary configuration within the scope of the presently disclosed subject matter.

In the above exemplary embodiment, the oxygen dissociation curve is modeled based on the Hill's equation is exemplified as the rule for mutually converting the values of the partial pressure of arterial oxygen and the arterial oxygen saturation of the subject 20. In addition, the mapping function obtained in the case where the index “P50” used in the model has the standard value is exemplified as the reference function. However, in a case where a model without using the index “P50” is adopted as the conversion rule, the reference function may be defined as a function that mutually converts a value of the arterial oxygen saturation and a value of the partial pressure of arterial oxygen that is obtained in a case where all of the partial pressure of arterial carbon dioxide, the pH of arterial blood, the body temperature, and the concentration of 2,3-DPG in blood have standard values.

The information illustrated in FIGS. 4, 5, and 7-10 need not be always visualized in the visualizing device 12. The information may be visualized in accordance with an appropriate instruction that is inputted through the user interface 13.

In the above exemplary embodiment, the processing device 11 is provided as a device independent of the pulse oximeter that measures the SpO2 of the subject 20. However, the processing device 11 may be incorporated in the pulse oximeter.

1. A processing device, comprising:
an interface configured to receive:
first data corresponding to a measured value of partial pressure of arterial oxygen and a measured value of arterial oxygen saturation of a subject; and
second data corresponding to a value of at least one of partial pressure of arterial carbon dioxide, pH of arterial blood, body temperature, and concentration of 2,3-DPG in blood of the subject; and
a processor configured to determine a rule for mutually converting values of the partial pressure of arterial oxygen and the arterial oxygen saturation of the subject based on at least one of the first data and the second data, and to cause a visualizing device to visualize the rule.

2. The processing device according to claim 1,
wherein the processor is configured to visualize the visualizing device to visualize the rule along with a reference function corresponding to the rule to be obtained in a case where a P50 that represents a value of the partial pressure of arterial oxygen obtained when the arterial saturation exhibits 50% has a standard value.

3. The processing device according to claim 1,
wherein the processor is configured to cause the visualizing device to visualize variable range of the rule in accordance with a variable range of the value corresponding to the second data.

4. The processing device according to claim 1,
wherein the processor is configured to cause the visualizing device to visualize, in response to reception of an assumed value of the value corresponding to the second data by the interface, the rule to be changed based on the assumed value.

5. The processing device according to claim 4,
wherein the assumed value is within a normal range of the value corresponding to the second data.

6. The processing device according to claim 5,
wherein the normal range is determined in accordance with attribute information of the subject.

7. The processing device according to claim 1,
wherein the processor is configured to perform, in response to reception of third data corresponding to transcutaneous arterial oxygen saturation of the subject by the interface, at least one of:
calculation of an estimate of the partial pressure of arterial oxygen based on the third data and the rule; and
calculation of an estimate of an index indicative of an oxygenation capability of the subject based on an estimate of the partial pressure of arterial oxygen and fourth data corresponding to fraction of inspired oxygen of the subject; and
wherein the processor is configured to cause the visualizing device to visualize the estimate as calculated.

8. The processing device according to claim 7,
wherein the processor is configured to cause the visualizing device to visualize the estimate of the partial pressure of arterial oxygen that is obtained by converting a value of the transcutaneous arterial oxygen saturation corresponding to the third data with a reference function corresponding to the rule to be obtained in a case where a P50 that represents a value of the partial pressure of arterial oxygen obtained when the arterial saturation exhibits 50% has a standard value.

9. The processing device according to claim 7,
wherein the processor is configured to cause the visualizing device to visualize marker indicative of an error in a measured value of the transcutaneous arterial oxygen saturation corresponding to the third data along with at least one of the rule and a reference function corresponding to the rule to be obtained in a case where a P50 that represents a value of the partial pressure of arterial oxygen obtained when the arterial saturation exhibits 50% has a standard value.

10. The processing device according to claim 7,
wherein the processor is configured to cause the visualizing device to visualize changes over time of the estimate.

11. The processing device according to claim 10,
wherein the processor is configured to cause the visualizing device to visualize changes over time of a variable range of the estimate in accordance with a variable range of the value corresponding to the second data.

12. The processing device according to claim 1,
wherein the processor is configured to perform an update of the rule based on least one of the first data and the second data, and to visualize the rule before the update and the rule after the update.

13. A non-transitory computer-readable medium having stored a computer program adapted to be executed by a processor installed in a processing device, the computer program being configured to cause, when executed, the processing device to:
receive first data corresponding to a measured value of partial pressure of arterial oxygen and a measured value of arterial oxygen saturation of a subject;
second data corresponding to a value of at least one of partial pressure of arterial carbon dioxide, pH of arterial blood, body temperature, and concentration of 2,3-DPG in blood of the subject;
determine a rule for mutually converting values of the partial pressure of arterial oxygen and the arterial oxygen saturation of the subject based on at least one of the first data and the second data; and
cause a visualizing device to visualize the rule.

14. A monitoring system, comprising:
a processing device; and
a visualizing device,
wherein the processing device includes:
an interface configured to receive:
first data corresponding to a measured value of partial pressure of arterial oxygen and a measured value of arterial oxygen saturation of a subject; and
second data corresponding to a value of at least one of partial pressure of arterial carbon dioxide, pH of arterial blood, body temperature, and concentration of 2,3-DPG in blood of the subject; and
a processor configured to determine a rule for mutually converting values of the partial pressure of arterial oxygen and the arterial oxygen saturation of the subject based on at least one of the first data and the second data, and to cause the visualizing device to visualize the rule.
